(19) United States
(12) Patent Application Publication
(10) Pub. No.: US 2025/0186028 A1
(43) Pub. Date: Jun. 12, 2025

(54) METHOD FOR PRODUCING A PRESSURE EQUALISATION AND ASSOCIATED SET

(71) Applicant: SARSTEDT AG & CO. KG

(72) Inventors: Mark Weinstock, Helmenzen (DE); Knut Kaspereit, Koeln (DE)

(21) Appl. No.: 18/981,458

(22) Filed: Dec. 14, 2024

Related U.S. Application Data
(63) Continuation of application No. PCT/EP2023/066581, filed on Jun. 20, 2023.

(30) Foreign Application Priority Data
Jun. 28, 2022 (DE) DE102022116057.2

(51) Int. Cl. A61B 10/00

(52) U.S. Cl. CPC A61B 10/0045

(57) ABSTRACT

A set including a container configured to store at least one bodily fluid; and a cover configured to close the container, wherein the container includes an interior configured to store the bodily fluid, wherein the set is transferrable at least between a sealing position and a venting position, wherein the cover and the container jointly form an airtight sealing seat in the sealing position of the set, wherein the cover includes an extraction unit configured to extract the bodily fluid from the container when the set is in the sealing position, wherein the cover and/or the container are deformable by a pressure differential between the interior of the container and an ambient when a vacuum is present in the interior of the container caused by the extraction of the bodily fluid by the extraction unit from the container.

### RELATED APPLICATIONS

This application is a continuation of international patent application PCT/EP2023/066581 filed on Jun. 20, 2023 claiming priority from German patent application DE 10 2022 116 057.2 filed on Jun. 28, 2022, both of which are incorporated in their entirety by this reference.

### FIELD OF THE INVENTION

The invention relates to a set for producing a pressure equalization. The invention also relates to a method for generating a pressure equalization.

### BACKGROUND OF THE INVENTION

The set includes at least one container and at least one cover configured to close the container. The container of the set includes an interior space where a bodily fluid is storable. The bodily fluid to be stored is in particular blood, urine, saliva, or another bodily fluid obtained from a patient. Typically, the bodily fluid is placed in the container after drawing and shall be stored in the container until analyzed. The cover of the set provides safe storage. The set is transferable at least between a sealing position and a venting position. The container and the cover jointly form an airtight seal in the sealing position of the set. Put differently, the cover is in contact with the container so that an outflow of air from the container is prevented. This also prevents an outflow of the bodily fluid from the interior of the container. The set is typically transferred into the sealing position after drawing the bodily fluid in order to provide safe transportation or safe storage of the bodily fluid.

Additionally, the set includes an extraction unit. The extraction unit enables extracting the bodily fluid from the interior of the container, even when the container is in the sealing position, thus the cover closes the container airtight. Using the extraction unit an amount of the bodily fluid that is to be analyzed is extracted from the container, wherein the extraction unit facilitates a particularly simple extraction of the bodily fluid from the container, since removal of the cover from the container is not required. Extracting the bodily fluid can thus be performed by an extraction device, e.g. a syringe, including an inserted cannula or not including a cannula, wherein the syringe is insertable through the extraction unit into the interior of the container in order to vacuum extract the bodily fluid from the container.

### BRIEF SUMMARY OF THE INVENTION

A generic set of the type described supra is already known in the art. Reference is made in particular to the “V-Monovette Urin” by applicant. Known sets for storing a bodily fluid, including an extraction unit, configured to extract the bodily fluid, however, have the problem that a pressure differential between an interior of the container and an ambient of the set is created due to the extraction of the bodily fluid from the container, e.g. by vacuum-extracting the bodily fluid using a syringe. The ambient pressure typically corresponds to atmospheric pressure. When reaching a particular level of vacuum, an additional vacuum extraction of the bodily fluid from the container is prevented since an inflow of air from the ambient into the interior of the container is prevented due to the airtight seal between the cover and the container. An inflow of air through the extraction unit is also prevented. In order to facilitate additional vacuum extraction of the bodily fluid, the container initially has to be vented. Thus, the set has to be transferred into a venting position. Thus, the cover of the set has to be removed from the container in order to let air flow into the interior of the container and thus equalize the pressure differential. Since the cover is typically screwed onto the container, this procedure has proven cumbersome and time-consuming. Additionally, the bodily fluid can be unintentionally spilled when opening the container again. Extraction of the bodily fluid can thus only be performed in portions and is interrupted by manually venting the container.

Thus, it is an object of the invention to provide a set which facilitates simple extraction of the bodily fluid from the container when the set is in the sealing position.

The object is achieved according to the invention by a set, including at least one container configured to store at least one bodily fluid; and at least one cover configured to close the container, wherein the container includes at least one interior configured to store the bodily fluid, wherein the set is transferrable at least between a sealing position and a venting position, wherein the cover and the container jointly form an airtight sealing seat in the sealing position of the set, wherein the cover includes an extraction unit configured to extract the bodily fluid from the container when the set is in the sealing position, wherein the cover and/or the container are deformable by a pressure differential between the interior of the container and an ambient when a vacuum is present in the interior of the container caused by the extraction of the bodily fluid by the extraction unit from the container, so that the set is transferrable from the sealing position into the venting position, wherein air from the ambient flows into the interior of the container and vents the interior of the container in the venting position of the set. Advantageous embodiments can be derived from the dependent claims.

The set is characterized in that the cover and/or the container is deformable by a pressure differential between the interior of the container and the ambient when a vacuum is present in the interior of the container, due to the extraction of the bodily fluid from the container, so that the set is transferrable from the sealing position into the venting position, wherein air from the ambient flows into the interior of the container and vents the interior of the container in the venting position of the set.

The set according to the invention has many advantages. In particular, the set facilitates extracting a bodily fluid from the interior of the container through an extraction unit, wherein removing the cover from the container is not required to facilitate pressure equalization. The set rather has a self-venting function. Put differently, manual interference is not required in order to vent the interior of the container. The cover and/or the container deform due to the extraction of the bodily fluid, e.g., in order to process and/or examine the bodily fluid, since the extraction causes a pressure differential between the interior of the container and an ambient due to the airtight sealing between the cover and the container. A reduction of the pressure within the container occurs due to the extraction of the bodily fluid from the container, so that a vacuum exists in the interior of the container. This causes the deformation of the container and/or the cover recited supra.

Thus, it is conceivable that the cover has an essentially flat cover base and cover rim, wherein the cover base deforms due to the vacuum in the interior of the container, so that the cover base is cambered. The set is transferred into the venting position due to the deformation of the cover and/or the container. The venting position of the set facilitates an inflow of air from the ambient of the set so that the interior of the container is vented. As soon as the pressure differential between the interior and of the ambient has been equalized, the set transitions into the sealing position again self-acting since the deformation of the cover and/or of the container only occurs due to the pressure differential which, however, was equalized after the venting. An inflow of air into the interior of the container is only facilitated when the set is in the venting position. In the sealing position, however, no inflow of air must occur. Advantageously, the set facilitates extracting any amount of bodily fluid from the container without having to manually vent the interior.

In addition to the sealing position and the venting position, the set can have other positions, in particular, a position where the cover is removed from the container. This position can be used to fill the bodily fluid into the container. The set can be transferred into the sealing position after filling in that the cover is placed in contact with the container, e.g., the cover is threaded onto the container.

Thus, the cover includes at least one venting element according to an advantageous embodiment of the invention, wherein the venting element generates at least one gap in the seal seat between the cover and the container when the set is in the venting position so that the gap is created due to the deformation of the cover and/or of the container due to a contact with the container and the cover so that air can flow from the ambient through the gap into the interior of the container. Advantageously, the venting element is pressed against the cover and/or the container due to the deformation of the cover and/or of the container. It is conceivable that the cover is deformed by the vacuum so that the cover base of the cover cambers wherein the cover rim is also deformed at least partially due to the deformation of the cover base and a deforming portion of the cover rim generates the gap in the seal seat. When the vacuum within the container increases, the gap that is being generated becomes larger. As a consequence, air from the ambient can flow into the interior of the container and vent the container. The inflowing air equalizes the pressure differential between the interior and the ambient. Advantageously, the set is transferred back into the starting position and the sealing position where the sealing seat is provided between the cover and the container due to the deformation of the cover and/or the container.

Advantageously, the venting element is configured as a cam. Tests have shown that configuring the venting element as a cam is particularly well suited to generate a gap in the seal seat between the cover and the container. The cam can thus be advantageously configured drop shaped and arranged at the cover of the set so that a wider side of the drop shaped cam comes in contact with the container and thus forms the gap when the cover is deformed.

Thus, according to an advantageous embodiment of the invention the cam is arranged at a bottom side of the cover that is oriented towards the container when the set is in the sealing position. The cover closes the container in the sealing position of the set and a bottom side of the cover is advantageously oriented towards the interior of the cover. When the cover and/or the container deforms, the distance between the cam and a wall of the container can be reduced so that the cam comes in contact with the wall of the container and forms the gap in the seal seat when the distance is reduced further. Arranging the cam at the bottom side of the cover has proven particularly advantageous in case the cover deforms due to the pressure differential. Thus, the cam should be arranged at the bottom side of the container, so that the cam is not in contact with the container in a closed condition of the container, this means when the cover is arranged on the container and the set is arranged in the sealing position. The cam should only come in contact with the container, in particular, its wall and transfer the set into the venting position when the vacuum is provided in the interior of the container. A threshold vacuum that causes the deformation of the cover can be predetermined by selecting a material of the cover and/or of the container. This ensures that the container does not deform already under a presence of a very small vacuum. A reforming of the cover into a starting position where the cam is not in contact with the container occurs after air flows from the ambient into the interior of the container. Thus, the set is provided in the sealing position again.

According to an advantageous embodiment, the extraction unit is configured tubular wherein the extraction unit is configured open at a first end and provided with a membrane that closes self-acting after piercing at a second end. Advantageously, the bodily fluid is extractable from the container by an extraction device, advantageously, a syringe that can include a cannula. Thus, a tubular configuration of the extraction unit is particularly advantageous, since the syringe is insertable into the container particularly easily when using a cannula since the extraction unit functions as a guide. Thus, the extraction unit advantageously extends directly down to the base of the container, which assures that the bodily fluid is extractable from the container even when a liquid level is very low. In any case, the extraction has to be performed so that the bodily fluid to be stored in the container does not exit from the container unintentionally during or after extraction. The self-closing membrane has proven particularly advantageous for this purpose since a manual interference of the user of the set is not required. The membrane closes the interior airtight relative to the ambient while being punctured and after being punctured so that no air can move into the interior of the container or in the opposite direction from the interior of the container into the ambient during extraction of the bodily fluid. By the same token, the self-closing membrane enables storing a liquid volume that has a liquid level that is above the second end. Put differently, the container can be filled with any amount of bodily fluid without the bodily fluid exiting from the container through the extraction unit.

According to an advantageous embodiment of the invention, the membrane includes a predetermined puncture location wherein the membrane is advantageously slotted in an area of the puncture location. Providing a predetermined puncture location assures that a user of the set does not have to puncture or punch through the membrane of the retrieval unit at a random location. Advantageously, the predetermined puncture location defines where the retrieval device has to be passed through. In case the membrane is slotted in the area of the puncture location, inserting the extraction device is additionally simplified since the extraction device then only has to be inserted through the slotted puncture location. Thus, it is advantageously provided that the slotted puncture location provides airtight and liquid tight sealing between the interior of the container and the ambient during and after the extraction by the extraction device.

A particularly advantageous embodiment of the method according of the invention provides that the container is cup-shaped. Typically containers that store bodily fluids are cup-shaped in order to provide simple handling. Thus, it can be advantageously provided that horizontal cross section of the container is enlarged in the longitudinal direction of the container. By the same token, it can also be provided that the cross section remains constant in the longitudinal direction.

Advantageously, the cover can have a circular cross section. Accordingly, the container can be configured beaker shaped and can also have a circular cross section so that the cover and the container are adapted to each other. According to an advantageous embodiment of the invention, the cover is supportable at the container by an attachment device, wherein the attachment device is advantageously configured as a thread. The thread facilitates attaching the cover at the container in a particularly quick, simple, and reliable manner. The thread assures in a simple manner that the bodily fluid stored in the container cannot run out. The extraction unit thus facilitates extraction from the container without having to unscrew the cover from the container.

It is particularly advantageous when a diameter of the cover is greater than a diameter of the container, so that the cover envelops the container at least partially in the sealing position of the set. When the cover includes a cover base and a cover rim, this embodiment is particularly advantageous since the cover base closes the container in particular in the sealing position and the cover rim envelops the container. Thus, it can be advantageously provided that the attachment device of the cover is arranged at an inner surface of the cover rim. By the same token, the container can also include an attachment device in a corresponding area. It is for example conceivable that the cover includes a thread that is threadable into a corresponding thread at an upper edge of the container so that the cover is threadable onto the container.

According to an advantageous embodiment of the invention, an opening cross section of the extraction unit is arranged in one plane with a cover surface, in particular, a bottom side of the cover. Put differently, the cover surface is configured flat. This way, the set is configured particularly compact and stackable easily.

According to another embodiment of the invention, the cover includes at least one air flow channel, wherein air can flow through the airflow channel from the ambient into the interior of the container after flowing through the gap. When the set is being used as intended, the cover can be threaded onto the container in the sealing position so that the cover does not contact a face of the container directly. Advantageously, the cover is only in contact with the container through the cover rim. Thus, a face of the container is not in contact with a bottom side of the cover. In case the cover is firmly threaded onto the container so that a contact between the face of the container and the cover base of the cover is established there is a risk that the container is not ventable since a positive form locking connection is provided between the cover base and the wall of the container. In this case, the airflow channel is particularly advantageous since the airflow channel facilitates air flowing into the interior of the container when the set is incorrectly assembled in the way described supra. The airflow channel is thus advantageously associated with a venting element so that air can initially flow through the airflow channel and thereafter through the gap formed by the venting element and thereafter into the interior of the container when the set is in the venting position.

According to an advantageous embodiment of the set according to the invention, it is furthermore provided that the airflow channel is arranged in a contact portion of the cover and of the container in the sealing position of the set, advantageously, in a circumferential annular face of the container. This arrangement facilitates a venting of the interior of the container in case the cover is arranged on the container so that an inflow is prevented due to a contact between the cover and the container in the contact portion.

The object recited supra is achieved by a method according to claim 14. An advantageous embodiment can be derived from the associated dependent claim.

The method facilitates providing a pressure equalization between an interior of a container of the set. The method includes the following steps: initially a bodily fluid and air are arranged in the interior of the container. Thus, the set is in the sealing position in which an exit of the bodily fluid and the air is prevented by a sealing seat between the cover and the container of the set.

The bodily fluid is extracted from the interior of the container in a second method step. The extraction unit of the set is used for this purpose. For example, the bodily fluid can be extracted from the interior of the container by an extraction device configured as a syringe. The extraction reduces a pressure that is provided in the interior of the container since an inflow of air compensating for the extraction of the bodily fluid is prevented by the sealing seat between the cover and the container. As a result, a vacuum over ambient is provided in the interior of the container. The cover and/or the container of the set are deformed by the vacuum.

A third method step transfers the set from the sealing position into the venting position due to the deformation wherein air flows from the ambient into the interior of the container in the venting position and vents the interior of the container. The set is returned into the sealing position as a result of the venting.

The advantages of the set according to the invention are analogously achieved by the method described supra.

An advantageous embodiment of the invention provides that the set is automatically transitioned between the sealing position and the venting position. Put differently, the transfer of the set between the positions occurs without manual interference. Thus, a user of the set can extract the bodily fluid from the container in a particularly simple manner without having to perform supplemental actions for venting the set since the set is self-venting. In particular, it is not necessary to remove the cover of the set during extraction of the bodily fluid in order to vent the interior of the container. The bodily fluid is extracted from the interior of the container without interruptions since the set is self-venting as soon as the pressure in the interior of the container goes below a threshold value. The threshold value can thus be advantageously predetermined by a selection of a material of the cover and/or of the container.

### BRIEF DESCRIPTION OF THE DRAWINGS

These set according to the invention and the method according to the invention are subsequently described based on an embodiment with reference to drawing figures, wherein:

FIG. 1 illustrates a three-dimensional view of a set according to the invention;

FIG. 2 illustrates a bottom side of a cover of the set according to FIG. 1;

FIG. 3A illustrates a detail view of the bottom side of the cover of FIG. 2, wherein the set is provided in a sealing position;

FIG. 3B illustrates a detail view of the bottom side of the cover of FIG. 2, wherein the set is provided in a venting position;

FIG. 4 illustrates a vertical sectional view of the set of FIG. 1;

FIG. 5 illustrates another sectional view of FIG. 1;

FIG. 6A illustrates a detail view of the sectional view of FIG. 5;

FIG. 6B illustrates another detail view of the sectional view of FIG. 5, wherein a venting channel of the set is open; and

FIG. 7 illustrates a vertical sectional view through an extraction unit of the set of FIG. 1.

### DETAILED DESCRIPTION

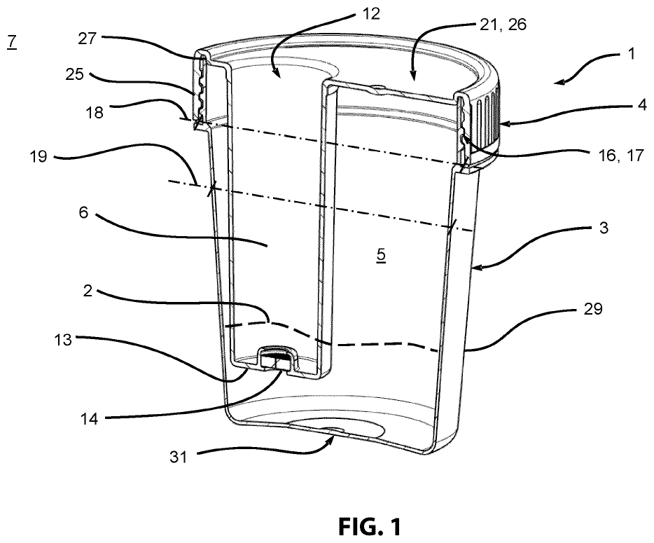

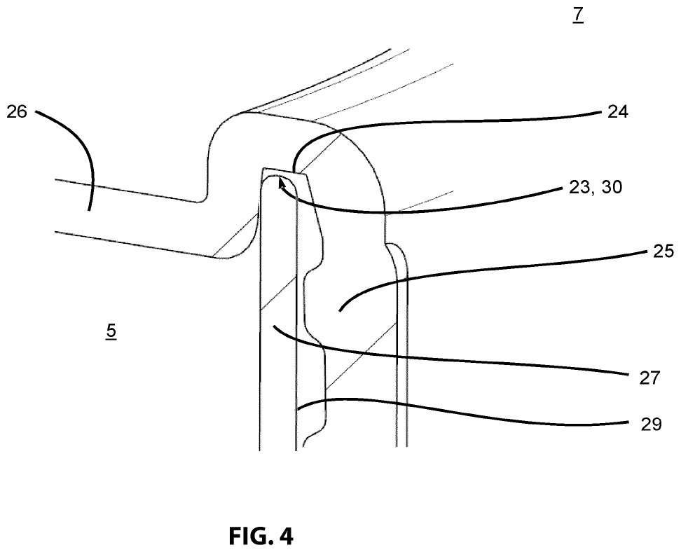

An embodiment illustrated in FIGS. 1-4 includes a set 1 according to the invention. The set is used for storing a bodily fluid 2, e.g. urine. The set 1 includes a container 3 for storing at least one bodily fluid 2 and a cover 4. The container 3 is configured beaker-shaped and has a circular cross-section that expands in a direction towards a topside 32 of the container 3. The cover 4 is configured to close the container 3 airtight. Thus, the cover 4 is also configured with a circular cross-section, wherein a diameter 18 of the cover 4 is greater than a diameter 19 of the container 3, so that the cover 4 is applicable to the container 3. Additionally, the cover 4 includes a cover rim 25 that extends perpendicular to a cover base 26 of the cover 4. This way the cover 4 envelops the container 3 at least partially when applied to the container 3.

The cover 4 and the container 3 respectively include an attachment device 16 configured as a screw thread 17 in order to be able to provide an airtight sealing seat between the cover 4 and the container 3. This way, the cover 4 is threadable onto the container 3 in a quick and simple manner. Thus, the screw thread 17 of the container 3 is arranged at an upper circumferential rim 27 at an outside of the container 3. The screw thread 17 of the cover 4 is arranged at an inside of the cover rim 25. The cover rim 25 reaches around the container 3 in and applied condition of the cover. The container 3 and the cover 4 are then transferrable into an airtight sealing position of the set 1 by threading the cover 4 onto the container 3 wherein and exit and inflow of air and an exit of the bodily fluid 2 from the inner space 5 of the container 3 is prevented in the airtight sealing position of the set 1.

The set 1 is transferrable between the sealing position and a venting position. Additionally, the set 1 can also be provided in a starting position where the cover 4 is separated from the container 3. The cover 4 is threaded onto the container 3 in the sealing position and an egress of the air or of the bodily fluid 2 is prevented. The sealing position is illustrated in FIG. 1.

The set 1 additionally includes an extraction unit 6. The extraction unit 6 facilitates an extraction of the bodily fluid 2 when the cover 4 is threaded on. The extraction unit is configured tubular, wherein a first end 12 of the extraction unit 6 is configured open, whereas the opposite second end 13 of the extraction unit 6 includes a membrane 14 which closes self-acting after being punctured. The membrane 14 as well as the sealing seat prevent an exit and an inflow of air from or into the interior space. The membrane 14 is impermeable for the bodily fluid 2. Thus, an opening cross section 20 of the extraction unit 6 is in an identical plane with a cover surface 21, namely, the bottom side 11 of the cover 4. The cover 4 is configured flat in spite of the extraction unit 6 being provided. An opening cross section 20 of the extraction unit 6 is arranged off center from a center point 28 of the cover 4 or the container 3, so that a small amount of the bodily fluid 2 is extractable by the extraction unit 6 from the container 3 while tilting the container 3.

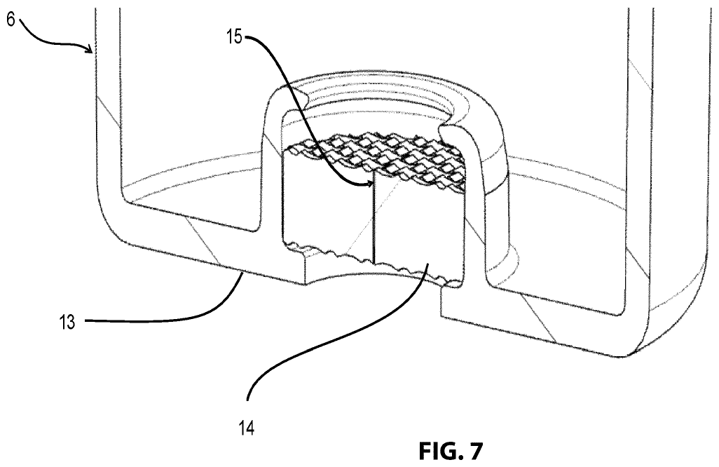

The extraction unit 6 is usable to extract a predetermined amount of the bodily fluid 2 from the container 3 for analysis. Thus, an extraction device, e.g., a syringe with a cannula or without a cannula is insertable through the membrane 14 into the bodily fluid 2. When reaching the membrane 14 arranged at an end of the extraction unit 6 the syringe is inserted through a predetermined puncture location 15 as evident in particular from FIG. 7. The bodily fluid 2 is extractable from the container 3 through the extraction unit 6 by a vacuum manually applied to the extraction device, in particular by drawing the syringe. The extraction device is thus easily insertable through the extraction unit 6 into the interior 5 with the cover 4 threaded on, wherein the self-closing membrane 14 assures that the bodily fluid 2 is separated airtight from the ambient 7 when the membrane 14 is punctured. The membrane 14 closes again self-acting after puncture. Thus, the sealing position is also maintained when the syringe is arranged in the interior 5.

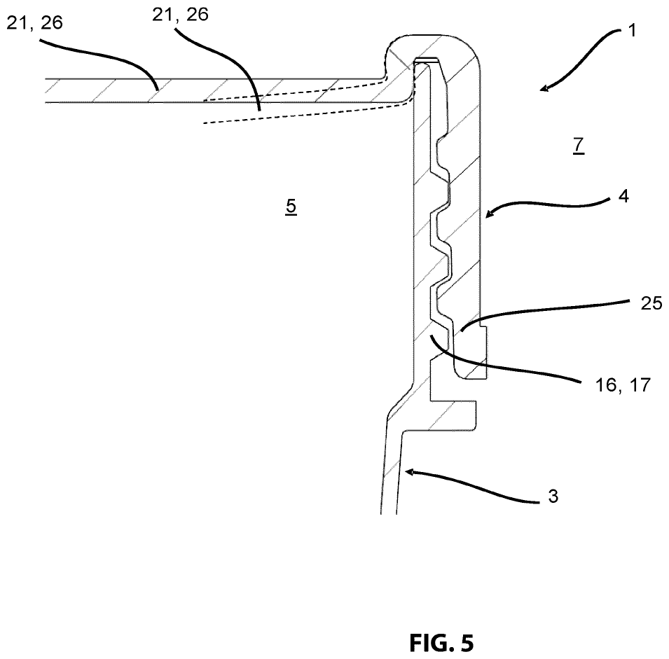

The syringe facilitates vacuum extracting the bodily fluid 2 from the interior 5 of the container and thus extracting a predetermined amount of the bodily fluid. A pressure prevailing in the interior 5 of the container 3 is reduced during vacuum extraction of the bodily fluid due to the airtight closure of the container 3 by the cover 4. A vacuum forms over the ambient 7 of the set 1 in the interior 5 of the container 3. The reduction of the pressure causes more and more difficulty for extracting the bodily fluid 2 from the container 3 since an inflow of air as a reaction to the retrieved bodily fluid 2 is prevented due to the sealing position of the set 1. When a certain level of vacuum is reached within the interior space 5, an additional vacuum extraction of the bodily fluid 2 is eventually rendered impossible. In order to prevent this, the cover 4 of the set 1 is deformed when reaching a predetermined pressure differential between the pressure in the interior 5 and atmospheric pressure in the ambient 7. In particular, the cover base 26 cambers in a direction towards a floor 31 of the container 3 as evident from FIG. 5. The pressure differential where the deformation of the cover 4 occurs is thus adaptable by selecting the material for the cover 4/or the container 3.

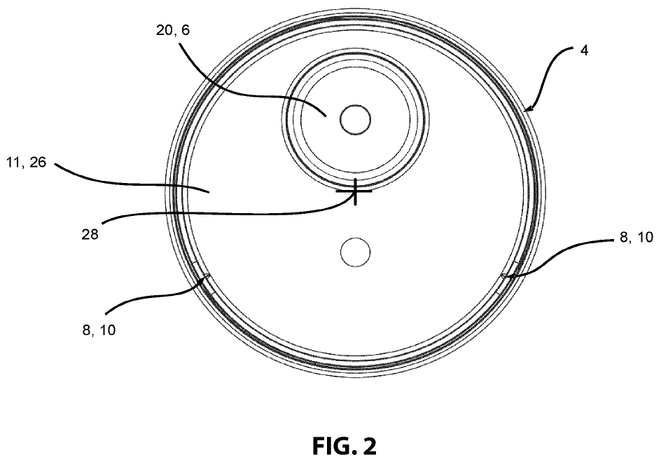

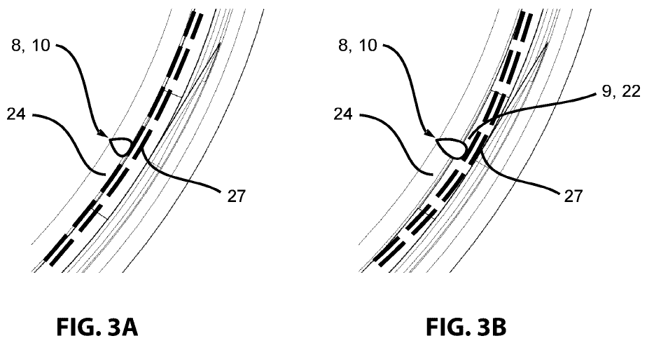

The cover 4 of the set 1 includes two venting elements 8 that are arranged at a bottom side 11 of the cover 4 in a rim portion of the cover 4. The venting elements 8 are respectively configured as a cam 10 as evident from FIGS. 2 and 3.

The cams 10 respectively contact a wall 29 of the container due to the deformation of the cover 4. This way, a gap 9 is formed between the cover 4 and the container 3 as evident from FIG. 3B. Thus, the set 1 is transferred from the sealing position into a venting position in which an inflow of air from the ambient 7 into the interior space of the container is facilitated. As a consequence, the interior 5 of the container 3 is vented and the vacuum is released so that an extraction of the bodily fluid 2 from the interior 5 is enabled again. The cover 4 deforms back into a starting position during the continuous pressure equalization due to the inflow of the air from the ambient 7 and the gap 9 is reduced in size. When the pressure equalization with the ambient 7 is completed, the gap 9 is closed completely and the set is in the sealing position again as evident from FIG. 3A. The deformation of the cover 4 and the associated forming of the gap 9 occurs automatically, meaning, without manual interference. Therefore, it is particularly simple for a user of the set 1 to vacuum extract the bodily fluid 2 from the interior 5 of the container 3 without having to provide a pressure equalization between the interior 5 of the container 3 and ambient 7 by removing the cover 4 from the container 3. Instead, the bodily fluid 2 is vacuum extracted from the interior 5 of the container 3 without the user of the set 1 detecting a change.

This process, thus transitioning between the sealing position and the venting position of the set 1 is repeated as a function of the volume of the bodily fluid 2 to be extracted. The amount of bodily fluid 2 that is extracted before the set 1 is automatically transitioned into the venting position essentially always stays the same. However, a user does not perceive the transition between the sealing position and the venting position since he does not have to interfere.

According to an advantageous embodiment, the cover 4 is threaded on so that a circumferential gap remains between a face 30 of the container 3 and the face 24 of the cover 4, so that the faces 24, 30 do not come in contact with one another. A contact shall only be provided in a portion of the two threads 17.

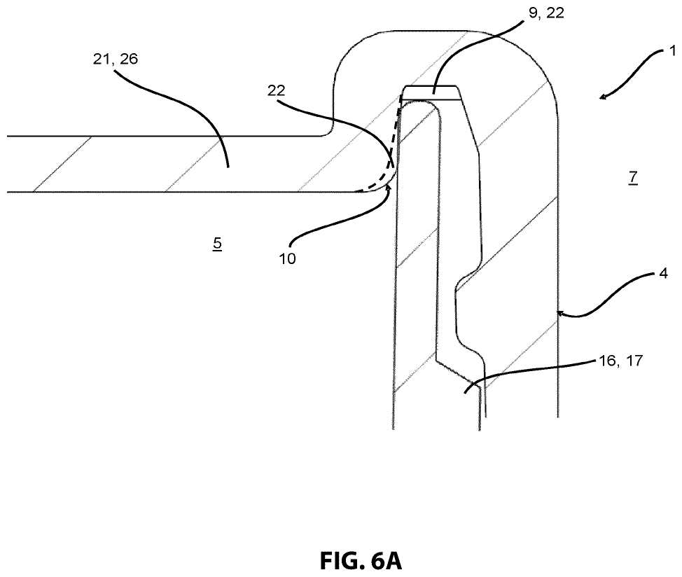

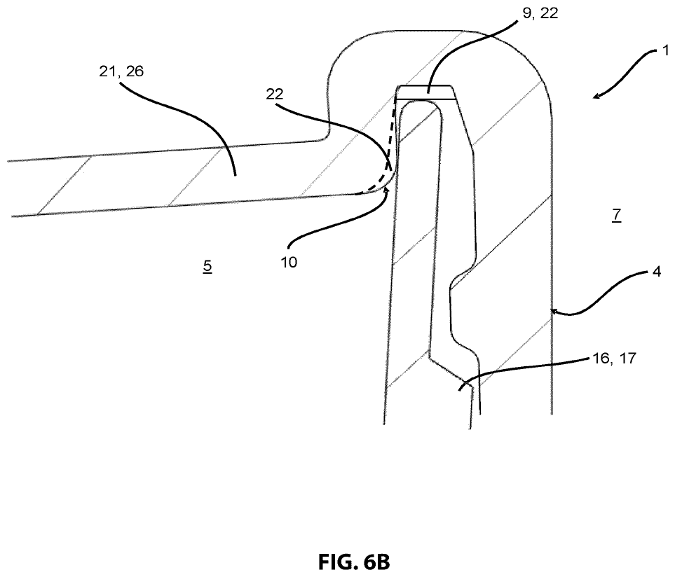

However, it has become evident that the cover 4 can be threaded on incorrectly so that the two faces 24, 30 come in contact with each other at least partially. This threaded connection is evident in particular from FIGS. 6A and 6B. In this embodiment, the interior 5 of the container 3 cannot be vented quickly enough even when the set 1 is in the venting position since an inflow of the air is impeded or prevented due to a contact of the two faces 24, 30. In order to prevent this from happening, an airflow channel 22 that is arranged at the face 24 of the cover 4 allows that air can flow into the interior 5 from the ambient 7 before flowing through the gap 9. For this purpose, the airflow channel 22 is arranged in a contact area 23 of the cover 4 and the container 3 in the sealing position of the set 1. Thus, the airflow channel 22 is associated with the respective cam 10. When the cover 4 is threaded onto the container 3 incorrectly, the set 1 can still be transferred into the venting position.

### REFERENCE NUMERALS AND DESIGNATIONS

1 set
2 bodily fluid
3 container
4 cover
5 interior
6 extraction unit
7 ambient
8 venting element
9 gap
10 cam
11 bottom side of cover
12 first end
13 second end
14 membrane
15 puncture location
16 attachment device
17 screw thread
18 diameter of cover
19 diameter of container
opening cross section of extraction unit
21 cover surface
22 airflow channel
23 contact portion
24 face of cover
25 cover rim
26 cover base
27 rim of container
28 center
29 wall
30 face of wall
31 floor of container
32 topside of container

What is claimed is:

1. A set, comprising:
a container configured to store at least one bodily fluid; and
a cover configured to close the container,
wherein the container includes an interior configured to store the bodily fluid,
wherein the set is transferrable at least between a sealing position and a venting position,
wherein the cover and the container jointly form an airtight sealing seat in the sealing position of the set,
wherein the cover includes an extraction unit configured to extract the bodily fluid from the container when the set is in the sealing position,
wherein the cover and/or the container are deformable by a pressure differential between the interior of the container and an ambient when a vacuum is present in the interior of the container caused by the extraction of the bodily fluid by the extraction unit from the container, so that the set is transferrable from the sealing position into the venting position, wherein air from the ambient flows into the interior of the container and vents the interior of the container in the venting position of the set.

2. The set according to claim 1,
wherein the cover includes at least one venting element that generates at least one gap in the sealing seat between the cover and the container when the set is in the venting position, and
wherein the gap is caused by a deformation of the cover and/or the container caused by a contact with the container and/or the cover, wherein air is able to flow from the ambient through the at least one gap into the interior of the container.

3. The set according to claim 2, wherein the venting element is configured as a cam.

4. The set according to claim 3, wherein the cam is arranged at a bottom side of the cover that is oriented towards the container when the set is in the sealing position.

5. The set according to claim 1,
wherein the extraction unit is configured tubular, and
wherein the extraction unit is configured open at a first end and provided with a membrane that is self-closing after puncture at a second end.

6. The set according to claim 5,
wherein the membrane includes a predetermined puncture location, and
wherein the membrane is slotted in an area of the predetermined puncture location.

7. The set according to claim 1, wherein the container is configured cup shaped.

8. The set according to claim 1, wherein the cover has a circular cross section.

9. The set according to claim 1, wherein the cover is supportable at the container by an attachment device configured as a screw thread.

10. The set according to claim 1, wherein a diameter of the cover is greater than a diameter of the container, so that the cover reaches around the container at least partially in the sealing position of the set.

11. The set according to claim 1, wherein an opening cross section of the extraction unit is arranged in a plane with a cover surface arranged at a bottom side of the cover.

12. The set according to claim 2,
wherein the cover includes at least one airflow channel, and
wherein air is able to flow from the ambient into the interior of the container through the at least one airflow channel before or after flowing through the at least one gap when the set is in the venting position.

13. The set according to claim 12, wherein the airflow channel is arranged in a contact portion of the cover and of the container in a circumferential annular face of the container in the sealing position of the set,

14. A method for generating a pressure equalization between an interior of a container of a set according to claim 1 and an ambient, the method comprising the steps:
arranging the set in the sealing position and arranging a bodily fluid and air in the interior of the container;
reducing a pressure in an interior of the container due to an extraction of a bodily fluid by the extraction unit from the interior so that a vacuum is generated in the interior of the container relative to an ambient of the set and deforming the cover and/or the container of the set,
wherein the deformation transfers the set from the sealing position into the venting position in which air flows from the ambient into the interior of the container and vents the interior of the container so that the set is transferred back into the sealing condition.

15. The method according to claim 14, wherein the set is automatically transferred between the sealing position and the venting position.
